(19) United States
(12) Patent Application Publication
(10) Pub. No.: US 2019/0357829 A1
(43) Pub. Date: Nov. 28, 2019

(54) PUSH- OR TWIST- INITIATED BLOOD METERING, FILTERING AND/OR STORAGE

(71) Applicant: Boston Microfluidics, Inc., Boston, MA (US)

(72) Inventors: Brian Weinberg, Boston, MA (US); Daniel Gussin, Boston, MA (US); Brandon T. Johnson, Somerville, MA (US); Catherine Fink, Boston, MA (US)

(21) Appl. No.: 16/173,101

(22) Filed: Oct. 29, 2018

Related U.S. Application Data
(60) Provisional application No. 62/675,870, filed on May 24, 2018.
(60) Provisional application No. 62/715,476, filed on Aug. 7, 2018.

(51) Int. Cl. A61B 5/15

(52) U.S. Cl. CPC A61B 5/150755; A61B 5/150282; A61B 5/150061; A61B 5/150343; A61B 5/150015; A61B 5/150305

(57) ABSTRACT

A device uses twist- or push-initiated force to collect, meter, filter and store a blood sample.

### CROSS REFERENCE TO RELATED APPLICATIONS

This application claims priority to a co-pending U.S. Provisional Patent Application Ser. No. 62/675,870 filed May 24, 2018 entitled “Push- Or Twist-Initiated Fluid Metering, Filtering and/or Storage”, and a co-pending U.S. Provisional Application Ser. No. 62/715,476 filed Aug. 7, 2018 entitled “Push- Or Twist-Initiated Blood Metering, Filtering and/or Storage”.

The entire contents of each of the above-referenced applications are hereby incorporated by reference.

### BACKGROUND

### Technical Field

This application relates to devices and methods for blood sample collection, metering, filtering and storage.

### Background Information

Blood used for diagnostic testing is usually extracted from a patient with a hypodermic needle and collected in a test tube. The collected blood is then packaged for shipment to a remote lab where various diagnostic tests are performed. However, many diagnostic tests require significantly less volume than the collected sample. Separation of cellular components from the sample is also needed for some tests.

Many tests only require small blood samples, where a finger stick rather than a hyperdermic needle can produce enough blood. But this small amount of blood cannot be easily transported to a lab. If the testing method cannot be immediately used at the same time the blood is extracted, a convenient reliable method of capturing, prepping, and preserving small amounts of blood is needed.

### SUMMARY

A device uses twist- or push-initiated force to collect, meter, filter and store a blood sample. The device includes a housing, a metering assembly, a filter, and blood storage media.

The metering assembly is disposed on top of the device and contains a well into which a blood sample is introduced. The well defines two regions—a lower region that provides a metered or defined volume of blood, and an upper region that accepts blood in excess of the metered portion. The bottom of the well is sealed with a pierceable membrane. A cap engages the well to provide a pushing or twisting (screwing) force to the metering assembly to force collected blood from the lower region into the upper region.

An absorbent element is located adjacent to and in fluid communication with the upper region.

In operation, the cap engages the well at a ledge located between the upper and lower regions of the well, to thereby force blood through one or more ports in the housing onto the absorbent element.

The pushing or twisting force provided by the cap also serves to rupture the pierceable membrane. In some embodiments, that rupturing can be provided by a post or other protrudion located on an inner surface of bottom of the housing.

The filter is positioned beneath the well, that is, below the pierceable membrane, and also provides fluid communication between the well and the storage media for the metered blood sample.

In some configurations, the metering assembly may contain a dry anticoagulant composition that is reconstituted when blood is introduced into the device.

The collection media may, in some implementations, be a separation media that separates plasma from whole blood in the metered blood sample.

The blood separation media preferably contains at least one region that can be to easily removed from the device for analysis.

### BRIEF DESCRIPTION OF THE DRAWINGS

The description below refers to the accompanying drawings, of which:

FIG. 1 is perspective view of the example device;

FIG. 2 is a longitudinal cross-section;

FIG. 3 is a transverse cross-section; and

FIG. 4 is a perspective view of a bottom cover.

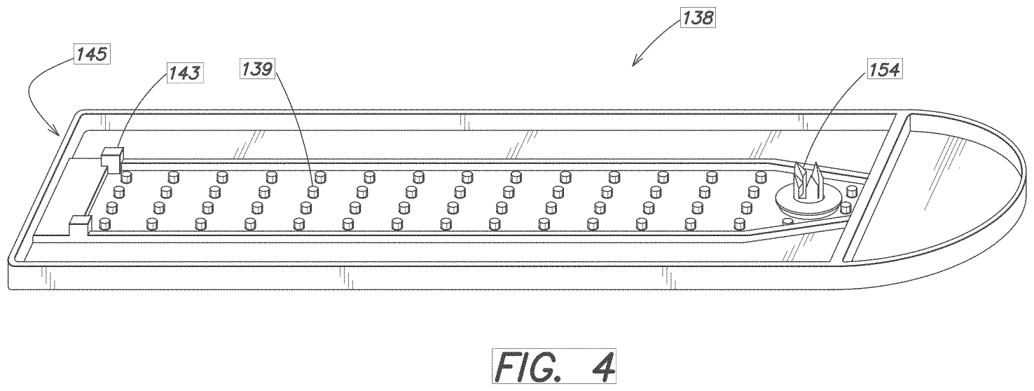

### DETAILED DESCRIPTION OF AN ILLUSTRATIVE EMBODIMENT

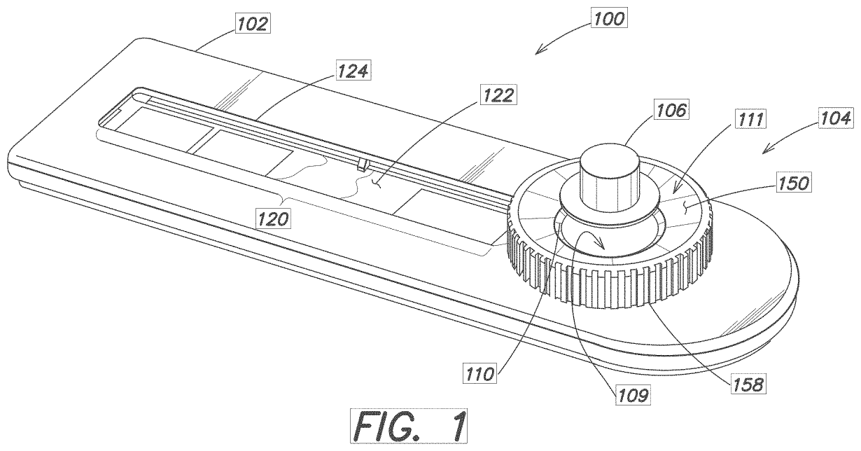

FIG. 1 is a perspective view of a device 100 for collecting and storing a metered amount of a blood sample. In one implementation, the device 100 is used to collect, store, and dry a blood sample for transport, such as to a remote laboratory for further analysis.

The device 100 generally consists of a housing or frame 102, a volume metering assembly 104, and a blood sample storage area 120.

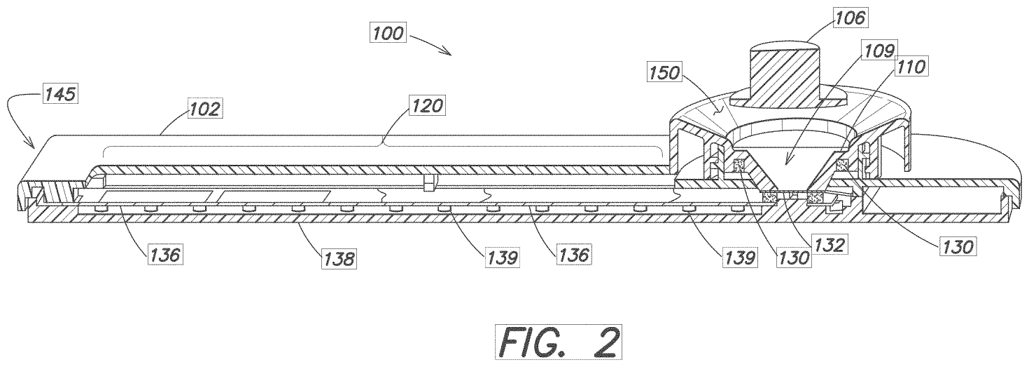

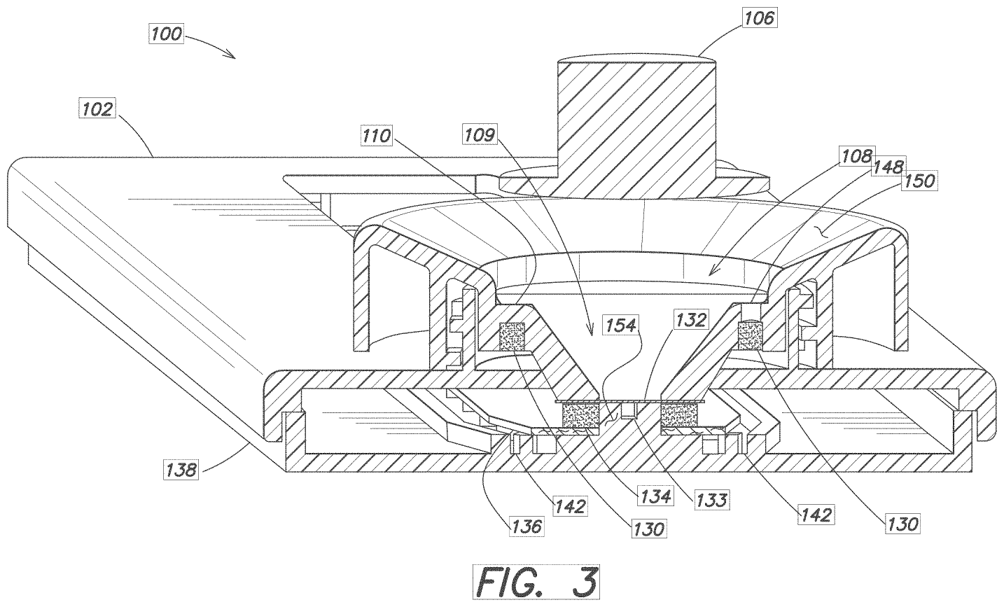

The volume metering assembly 104 consists of cap or hat 106 that engages a collection well 108 disposed above or on the frame 102. A Polyvinyl Alcohol (PVA) foam or other blood absorptive material (shown in FIGS. 2 and 3) is preferably fixed adjacent the well 108, such as at or near the bottom rim of the cap 106. The collection well 108 consists of two regions, a lower region 109 that in this embodiment is generally cylindrical in shape, and an upper region 111 defined by a tapered flange 150. A circumferential ledge 110 is located within the well 108 at a determined distance down from the flange 150. The lower region 109 has a defined volume for holding a metered amount of blood. The upper region 111 is used to collect a volume of blood in excess of the defined volume.

The storage area 120 contains a storage media 122 and an optional window 124 for viewing the collected blood sample. Suitable storage media 122 are described in more detail below.

FIG. 2 and FIG. 3 are longitudinal and transverse cutaway views showing the device 100 in more detail. The lower region 109 of the well 108 is wider at the top than at its bottom. A foam ring 130 or other absorbtive material is placed beneath the ledge 110 and surrounds the well. A foil or other pierceable material layer 132 either defines the bottom of the well, or is disposed around or near an opening 133 in the bottom of the well 108.

Also visible in FIGS. 2 and 3 is a filter media 134 disposed beneath or surrounding the pierceable material layer 132 at the bottom of the well 108. The filter 134 may be a media such as cotton, or a a synthetic sponge essentially composed of Polyvinyl Alcohol (PVA) or other open-celled, highly absorbent porous foam that wicks aqueous solutions. The filter 134 serves to control the flow of blood exiting the well 108 and flowing towards the storage area 120.

A storage media 122 such as a sucrose treated paper 136 is disposed within and supported by the frame 102 within the storage area 120. The storage media 122 may be a microfluidic separation membrane capable of separating blood plasma from whole blood. Other types of storage media 122 or treated papers 136 suitable for drying and storing blood may be used. One end of the paper 136 is placed adjacent the bottom of the well 108, typically at the exit point of the filter media 134; the other end of the paper 136 extends to the far end 145 of the frame 102. A bottom cover section 138 supports the paper 136 and may have a series of pegs 139 spaced apart from one another and/or ledges 143 to further support and hold the paper 136 in position.

Channels 142 formed in the frame 102 near the filter 134 may also support the paper 136 and/or direct a collected blood sample onto the paper 136.

The device 100 may be shipped with a peelable label or other protective cover (not shown) fixed over or within the well 108.

In operation, a caregiver or patient peels off the protective label or cover (if present) to expose the open well 108. They then stick their finger and drop blood into the well 108. Enough blood should be dispensed from the finger stick to fill the well 108 beyond the ledge 110 but not so much blood as to reach beyond the flange 150. By adding enough blood to fill up beyond that ledge 110, there is at least a minimum, metered, defined volume collected within the lower portion 109 of the well 108, in the area between the ledge 110 and the bottom foil 132. Defined small volumes from about 50 microliters (uL) to 300 microliters (uL) are typical.

The cap 106 is then dropped down to engage the device 100, such as via the inner (lower) rim of the flange 150. The cap is then pushed down or twisted to provide a positive force to close the well 108 and close off the defined volume in the lower tapered portion 109. If the cap 106 is a twist cap, internal threaded portions further encourage the cap 106 to close off the well 108 and provide positive mechanical force.

The twist or pushing action also pushes blood in excess of the defined volume, that is the blood located in the upper portion 111 into the surrounding foam 130 located underneath the ledge. In some embodiments, the excess blood may flow through one or more channels or ports 148 located around the outer periphery of the well 108 into the foam 130. In some embodiments, a ring shaped foam 130 may also be located around the periphery of the cap to further help to collect the excess blood.

The force of pressing down or twisting on the cap 106 also breaks the foil 132 on the bottom of the well 108. Such rupturing of the foil may be encouraged by one or more posts or protrusions 154 located in the bottom 138 of the frame 102. Blood then starts to flow towards the paper strip 136, through the filter material 134. The filter material may control how fast the blood flows to the paper 136.

The filter material 134 may also act as a compliant member, so that when the cap 106 is pushed or twisted down, it further assists with maintaining closure at the bottom of the well 108.

Once the blood reaches the paper 136, it continues to flow laterally away from the well 108 towards end 145. If the paper is a separation media, plasma may be separated from whole blood as the paper 136 wicks the blood away from the filter.

With the cap 106 firmly in place, the device is thus sealed for transport to a remote laboratory.

The device 100, including the bottom 138 or other parts of the frame 102 or other components should be easily disassembled so that the lab can access the stored blood and/or plasma sample on the paper 136. The paper 136 may be removable from the frame 102 so that a lab may cut it up, punch holes in it, or otherwise process it.

Additional design details are possible. For example, ribs 158 may be provided on outer rim of the the cap 106, to provide a greater area to enable the user to grip and/or twist the cap 106.

An anti-coagulant, such as a dry composition anti-coagulant, may be stored within the metering assembly 104 and activated when blood is placed in the well.

Ledge 110 around the periphery of the well 108 may also be particularly sized to define the overall outer diameter of the cap 106 independent of the volume defined by the lower portion 109.

What is claimed is:

1. A blood sample collection device comprising:
a housing;
a metering component comprising a collection well and a cap,
the collection well having an opening at a top and a pierceable membrane located adjacent a bottom, the collection well further defined by a lower region having a defined volume for collecting a metered portion of the blood sample, and an upper region for accepting an excess portion of the blood sample in excess of the defined volume, and
the cap engaging the upper region of the well, and providing twist- or push-initiated mechanical force on the well, and also piercing the membrane in response to the mechanical force;
absorptive media, disposed adjacent and in fluid communication with the upper region of the well, for storing the excess portion of the blood sample in excess of the defined volume;
a filter, disposed adjacent the pierceable membrane, for filtering the metered portion of the blood sample to provided a filtered blood sample; and
a storage media, disposed within the housing adjacent the filter, for storing the the filtered blood sample.

2. The device of claim 1 additionally wherein the storage media is disposed below the pierceable membrane.

3. The device of claim 2 additionally wherein the defined volume is further defined by a ledge formed on the periphery of the well.

4. The device of claim 3 additionally wherein fluid communication between the upper region of the well and the absorptive media is provided by one or more ports formed in the housing adjacent the ledge.

5. The device of claim 4 additionally wherein the cap engages the ledge for providing the twist- or push-initiated mechanical force.

6. The device of claim 1 additionally wherein the storage media is a microfluidic separation media for separating plasma from whole blood in the blood sample.

7. The device of claim 6 additionally comprising one or more supports for supporting the microfluidic separation media within the housing.

8. The device of claim 1 additionally comprising:
an anticoagulant disposed within the metering assembly.

9. The device of claim 1 additionally comprising:
a post located on the housing below the well adjacent the pierceable membrane, and positioned to rupture the pierceable membrane upon application of the mechanical force.

10. The device of claim 1 wherein the storage media is removable from the housing after application of the mechanical force.

11. The device of claim 1 additionally comprising:
a removable protective layer disposed on the collection well.

12. A device for collecting a small volume blood sample, comprising:
(a) a multipart housing comprised of a first part connected to a second part to define an interior space;
(b) a metering assembly disposed on the first part of the housing, wherein the metering assembly is configured to receive a small volume of a blood sample from a patient and deliver a smaller defined volume of blood elsewhere inside the housing, wherein the metering assembly is moveably retained in the first part of the housing such that it can be moved into the housing by application of force by a user and comprises (i) a well accessible to the user through an open top and comprising an upper region and a lower region, wherein the upper region is bounded by a first well wall and a lower flange and the lower region is bounded by a second well wall extending from the lower flange and a bottom opening sealed with a pierceable membrane, wherein the volume defined by the lower region defines the smaller defined volume to be delivered by the metering assembly and wherein the pierceable membrane that seals the bottom of the well is positioned proximate to a post protruding from the interior surface of the second part into the interior space of the housing such that when the metering assembly is moved into the interior space of the housing a sufficient distance the post can rupture the pierceable membrane, (ii) at least one port with an opening in or adjacent to the lower flange and which provides a flow path from the upper region of the well to an overflow region that comprises absorptive media, and (iii) a dry anticoagulant composition configured for reconstitution upon addition of a blood sample to the well;
(c) a cap configured for insertion into the upper region of the well and to sealingly engage the lower flange of the upper region, wherein by moving the cap to engage the lower flange, blood in the upper region of the well is forced through the port(s) in the lower flange into the absorptive media in the overflow region of the metering assembly;
(d) a filter disposed around the post and, when the pierceable membrane is ruptured by the post, in fluid communication with the lower region of the well; and
(e) a blood separation media disposed in the interior space of the housing in fluid communication with the filter, wherein the blood separation media comprises at least one removable sample region.
